(19) United States
(12) Patent Application Publication
(10) Pub. No.: US 2022/0370668 A1
(43) Pub. Date: Nov. 24, 2022

(54) NETWORK-CONNECTED STORAGE LOCKER STERILIZATION FOR EXCHANGE OF ITEMS

(71) Applicant: AT&T Intellectual Property I, L.P., Atlanta, GA (US)

(72) Inventors: Robert T. Moton, JR., Alpharetta, GA (US); Adrianne Binh Luu, Atlanta, GA (US); James Pratt, Round Rock, TX (US); Barrett Kreiner, Woodstock, GA (US); Walter Cooper Chastain, Atlanta, GA (US); Ari Craine, Marietta, GA (US); Robert Koch, Peachtree Corners, GA (US)

(21) Appl. No.: 17/327,059

(22) Filed: May 21, 2021

(51) Int. Cl. A61L 2/24; G07F 17/12; G07C 9/00; A61L 2/10; A61L 2/20; A61L 2/07; A61L 2/14; A61L 2/12; A61L 2/08

(52) U.S. Cl. CPC A61L 2/24; G07F 17/12; G07C 9/00571; G07C 9/00912; A61L 2/10; A61L 2/202; A61L 2/206; A61L 2/07; A61L 2/14; A61L 2/12; A61L 2/085; A61L 2202/122

(57) ABSTRACT

A processing system including at least one processor may obtain information regarding an exchange of an item, the exchange having a sterilization requirement for the exchange, and may identify a network-connected storage locker having at least one sterilization component that is capable of fulfilling the sterilization requirement and that is available for assignment to the exchange. The processing system may then transmit a first notification to the network-connected storage locker of a reservation of the network-connected storage locker for the exchange and transmit to a computing system associated with a source of the item a second notification of the reservation of the network-connected storage locker for the exchange.

The present disclosure relates generally to secure delivery systems, and more particularly to methods, computer-readable media, and apparatuses for reserving a network-connected storage locker having at least one sterilization component that is capable of fulfilling a sterilization requirement and that is available for assignment for an exchange of an item.

### BACKGROUND

As commerce continues to proliferate, especially Internet commerce, an ever increasing amount of commerce involves the delivery of products as packages to homeowners who are often unavailable to receive the packages in person due to obligations such as work. Thus, packages (especially large packages that do not fit in a mailbox) are often left at the door of the residence. Storage lockers are offered by various delivery services and merchants which customers may choose to utilize to receive items that are purchased. For instance, a customer may buy online and choose “pick-up” in store, where the item may be picked-up from a locker designated for the customer's item (e.g., rather than from a customer service counter).

### SUMMARY

In one example, the present disclosure describes a method, computer-readable medium, and apparatus for reserving a network-connected storage locker having at least one sterilization component that is capable of fulfilling a sterilization requirement and that is available for assignment for an exchange of an item. For example, a processing system having at least one processor may obtain information regarding an exchange of an item, the exchange having a sterilization requirement, and may identify a network-connected storage locker having at least one sterilization component that is capable of fulfilling the sterilization requirement and that is available for assignment to the exchange. The processing system may then transmit a first notification to the network-connected storage locker of a reservation of the network-connected storage locker for the exchange and transmit to a computing system associated with a source of the item a second notification of the reservation of the network-connected storage locker for the exchange.

### BRIEF DESCRIPTION OF THE DRAWINGS

The teachings of the present disclosure can be readily understood by considering the following detailed description in conjunction with the accompanying drawings, in which:

FIG. 1 illustrates an example system related to the present disclosure;

FIG. 2 illustrates an example exchange record and an example network-connected storage locker record, in accordance with the present disclosure;

FIG. 3 illustrates a flowchart of an example method for reserving a network-connected storage locker having at least one sterilization component that is capable of fulfilling a sterilization requirement and that is available for assignment for an exchange of an item; and

FIG. 4 illustrates a high level block diagram of a computing device specifically programmed to perform the steps, functions, blocks and/or operations described herein.

To facilitate understanding, identical reference numerals have been used, where possible, to designate identical elements that are common to the figures.

### DETAILED DESCRIPTION

Examples of the present disclosure describe methods, computer-readable medium, and apparatuses for reserving a network-connected storage locker having at least one sterilization component that is capable of fulfilling a sterilization requirement and that is available for assignment for an exchange of an item. For instance, examples of the present disclosure provide a network of network-connected storage lockers that enables items to be securely exchanged within a community and with enhanced antimicrobial/sterilization services. The items may be exchanged by sharing or in a marketplace arrangement. The community may be a neighborhood or other closed user groups, such as members of an electronic marketplace. For instance, a sharing arrangement may comprise a community pantry or the like, where members within a physical community such as a neighborhood might use network-connected storage lockers to freely (or via subscription or membership fees) share produce items grown in gardens, books, or other shareable goods.

To illustrate, a first party may be a user who has an item, such as a coffee maker, to sell, and who may be equipped with a networked device such as a smartphone. A second party may be similarly equipped. There may also exist a shared storage facility. This may be a shared network-connected storage locker facility that has one or more networked control modules that control(s) access to one or more locker units. Thus, each network-connected storage locker may be a fixed or relatively fixed item in an environment. An item, such as the coffee maker, may be identified for exchange. The parties may have separately arranged for the exchange, including negotiating a price and payment. A record of the arrangement may be sent to an exchange database. The record may include location information for the parties. In one example, the first party may optionally use an application on a user device to scan a code on the item and/or to enter information about the item.

In one example, the first party may also use a camera of the user device to capture one or more images of the item, wherein the application may estimate the dimensions of the item and record the dimensions in the exchange database. A processing system of the present disclosure (e.g., a network-based server, or servers) may use the dimensions and the locations of either or both parties to search for available network-connected storage lockers to identify an exchange point. For instance, the exchange point may be determined, at least in part, by the dimension requirements of the item. For example, the dimensions may be used to find a network-connected storage locker that is vacant and of sufficient size. In addition, a unit location that is most equidistant from both parties may be used, a location that is closest to the recipient party may be used, etc.

Either party may optionally specify one or more capabilities required for the network-connected storage locker. For example, if the item is sensitive to environmental condition such as heat or cold, a climate controlled unit may be requested. Likewise, a network-connected storage locker may be requested that has one or more sanitizing/antimicrobial components for sanitizing the item, e.g. ultraviolet light sanitizing capabilities, ozone sanitizing capabilities, etc. Alternatively, or in addition, a network-connected storage locker with a networked camera may be requested to display the item once it is placed in the unit.

In one example, the present disclosure may enable the item to be deposited in a network-connected storage locker for an unidentified receiving party. For instance, the first party may wish to sell or make available the item, but does not have a buyer or another party to receive the item at the moment. In this case, the item may be considered to be renting the storage unit while it is available for acquisition at a future time. The first party may request a network-connected storage locker based on just the dimensions of the item. The first party may also specify features for the network-connected storage locker to be selected such as a camera, sanitization capabilities, climate control, etc. In this case, the item may be advertised, for instance on a website or via an app, as being available for acquisition. The advertised listing may include a live feed from the camera, a price, and other information about the item.

If the receiving party is unidentified, the first party may, in the course of requesting a storage unit, request the server(s) to predict a best location for the network-connected storage locker. The server(s) may access data from one or more sources to help make this prediction. For example, the server may access historical purchase data, wishlist data, and the like. This may be data from a membership database for an online merchant membership club, or the like. In this way, the processing server(s) may predict where the item is most likely be sold. In one example, the present disclosure may push an alert to a potential buyer or another party who is located nearby to alert that an item on a wishlist is nearby.

After the storage unit is identified, a unit ID and location may be sent to a user device of the first party. In addition, a status of the network-connected storage locker may be marked as “reserved” in a network-connected storage locker database. In one example, a unique key is sent to a user device of the first party to access the network-connected storage locker, e.g., a quick response (QR) code, a password/passcode, or the like. Thus, for example, the first party may access the network-connected storage locker by scanning the QR code at a scanner of the network-connected storage locker, entering a passcode via a keypad of the network-connected storage locker, etc. to open a door, may place the item in the network-connected storage locker, and may secure the network-connected storage locker and the item therein by closing the door. The network-connected storage locker may transmit a notification of the accessing of the network-connected storage locker to the server(s), which may change the status of the network-connected storage locker in the network-connected storage locker database to “in use.” The server(s) may send a message to the second party (or to one or more potential buyers or other interested parties) that the item is available to acquire. The first party may have specified that the network-connected storage locker be capable of sterilizing the surface of the item, in which case a UV light or other sterilization components may be deployed by the network-connected storage locker after the first party secures the network-connected storage locker by closing the door.

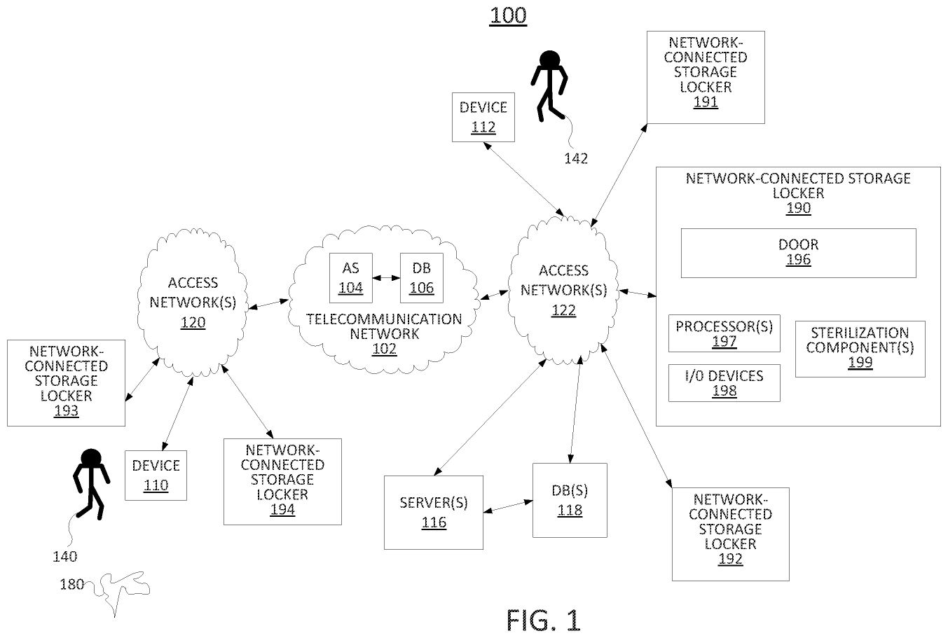

A second party identified for the exchange of the item (either prior to deposit in the network-connected storage locker or at some future time after the item is placed therein) may receive a one-time key, such as a QR code, password/passcode, or the like, and may use it to access the network-connected storage locker and retrieve the item. An in-unit camera may be used to view the interior and to confirm that the network-connected storage locker is empty. For instance, one or more images may be transmitted to the server(s), which may analyze the image(s) to confirm that the network-connected storage locker is empty, whereupon the server may change the status of the network-connected storage locker in the network-connected storage locker database to “vacant.” In one example, the network-connected storage locker may self-sterilize using an ozone source, a UV light, or other available sanitization components. Upon detection that the item was retrieved, a record of the transaction made be made, including an identifier for the party retrieving the item, a timestamp, a location stamp, and other data such as to be used for billing or other purposes. These and other aspects of the present disclosure are described in greater detail below in connection with the examples of FIGS. 1-4.

To aid in understanding the present disclosure, FIG. 1 illustrates an example system 100 in which examples of the present disclosure for reserving a network-connected storage locker having at least one sterilization component that is capable of fulfilling a sterilization requirement and that is available for assignment for an exchange of an item may operate. The system 100 may include any one or more types of communication networks, such as a circuit switched network (e.g., a public switched telephone network (PSTN)) or a packet network such as an Internet Protocol (IP) network (e.g., an IP Multimedia Subsystem (IMS) network), an asynchronous transfer mode (ATM) network, a wireless network, a cellular network (e.g., 2G, 3G, 4G, 5G and the like), a long term evolution (LTE) network, and the like, related to the current disclosure. It should be noted that an IP network is broadly defined as a network that uses Internet Protocol to exchange data packets. Additional example IP networks include Voice over IP (VoIP) networks, Service over IP (SoIP) networks, and the like.

In one example, the system 100 may comprise a telecommunication network 102. The telecommunication network 102 may be in communication with one or more access networks 120 and 122, and the Internet (not shown). In one example, telecommunication network 102 may combine core network components of a cellular network with components of a triple play service network; where triple-play services include telephone services, Internet services and television services to subscribers. For example, telecommunication network 102 may functionally comprise a fixed mobile convergence (FMC) network, e.g., an IP Multimedia Subsystem (IMS) network. In addition, telecommunication network 102 may functionally comprise a telephony network, e.g., an Internet Protocol/Multi-Protocol Label Switching (IP/MPLS) backbone network utilizing Session Initiation Protocol (SIP) for circuit-switched and Voice over Internet Protocol (VoIP) telephony services. Telecommunication network 102 may further comprise a broadcast television network, e.g., a traditional cable provider network or an Internet Protocol Television (IPTV) network, as well as an Internet Service Provider (ISP) network. In one example, telecommunication network 102 may include a plurality of television (TV) servers (e.g., a broadcast server, a cable head-end), a plurality of content servers, an advertising server, an interactive TV/video on demand (VoD) server, and so forth. For ease of illustration, various additional elements of telecommunication network 102 are omitted from FIG. 1.

In one example, the access networks 120 and 122 may comprise Digital Subscriber Line (DSL) networks, public switched telephone network (PSTN) access networks, broadband cable access networks, Local Area Networks (LANs), wireless access networks (e.g., an Institute for Electrical and Electronics Engineers (IEEE) 802.11/Wi-Fi network and the like), cellular access networks, 3^{rd} party networks, and the like. For example, the operator of telecommunication network 102 may provide a cable television service, an IPTV service, or any other types of telecommunication service to subscribers via access networks 120 and 122. In one example, the access networks 120 and 122 may comprise different types of access networks, may comprise the same type of access network, or some access networks may be the same type of access network and other may be different types of access networks. In one example, the telecommunication network 102 may be operated by a telecommunication network service provider. The telecommunication network 102 and the access networks 120 and 122 may be operated by different service providers, the same service provider or a combination thereof, or may be operated by entities having core businesses that are not related to telecommunications services, e.g., corporate, governmental or educational institution LANs, and the like.

In one example, the access networks 120 may be in communication with one or more devices, e.g., device 110, network-connected storage lockers 193, 194, etc. Similarly, access networks 122 may be in communication with one or more devices, e.g., device 112, network-connected storage lockers 190-192, server(s) 116, database(s) (DB(s)) 118, etc. Access networks 120 and 122 may transmit and receive communications between devices 110 and 112, server(s) 116, network-connected storage lockers 190-194, and components of telecommunication network 102, devices reachable via the Internet in general, and so forth. In one example, each of the devices 110 and 112 may comprise any single device or combination of devices that may comprise a user endpoint device. For example, the devices 110 and 112 may each comprise a mobile device, a cellular smart phone, a laptop, a tablet computer, a desktop computer, an application server, a bank or cluster of such devices, and the like.

In one example, the access networks 122 may also be in communication with one or more servers 116 and one or more databases (DBs) 118. The server(s) 116 and DB(s) 118 may comprise or be associated with, for example, a network-connected storage locker management system and/or reservation system. In one example, DB(s) 118 may comprise physical storage device(s) integrated with server(s) 116 (e.g., a database servers), or attached or coupled to the server(s) 116. For instance, DB(s) 118 may store and provide one or more user databases, network-connected storage locker databases, item exchange/transaction databases, and so forth.

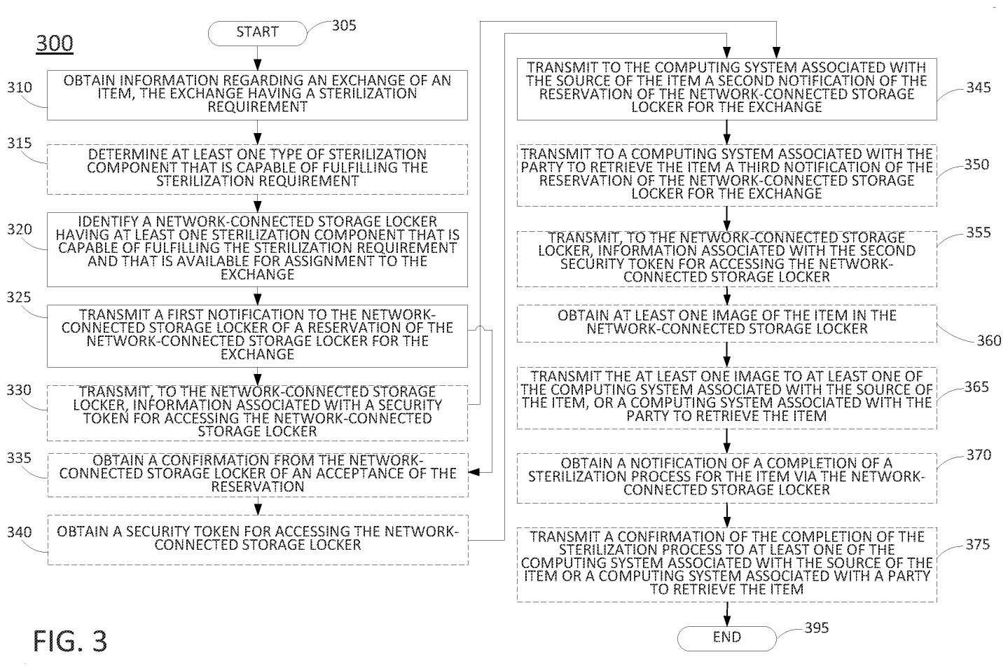

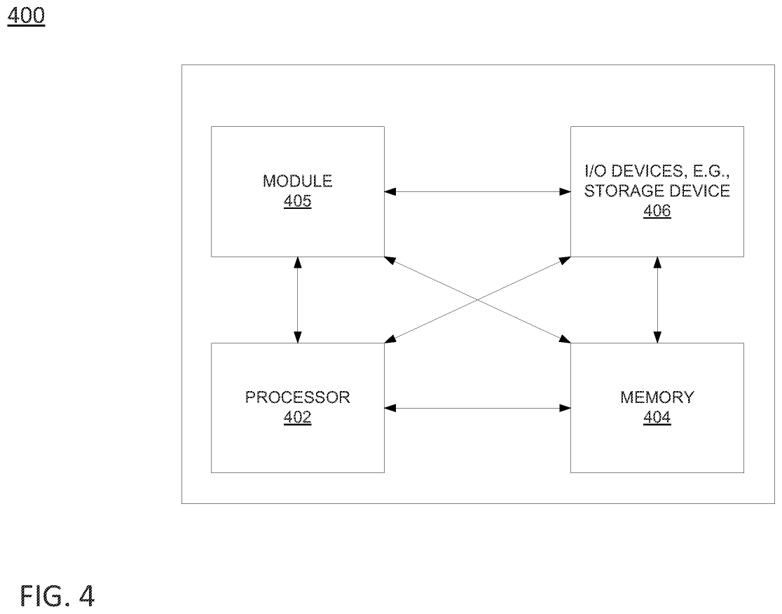

In one example, each of server(s) 116 may comprise a computing device or processing system, such as computing system 400 depicted in FIG. 4, wherein one or more of the server(s) 116 may be configured to perform one or more steps, functions, or operations for reserving a network-connected storage locker having at least one sterilization component that is capable of fulfilling a sterilization requirement and that is available for assignment for an exchange of an item. For instance, an example method for reserving a network-connected storage locker having at least one sterilization component that is capable of fulfilling a sterilization requirement and that is available for assignment for an exchange of an item is illustrated in FIG. 3 and described below. In addition, it should be noted that as used herein, the terms “configure,” and “reconfigure” may refer to programming or loading a processing system with computer-readable/computer-executable instructions, code, and/or programs, e.g., in a distributed or non-distributed memory, which when executed by a processor, or processors, of the processing system within a same device or within distributed devices, may cause the processing system to perform various functions. Such terms may also encompass providing variables, data values, tables, objects, or other data structures or the like which may cause a processing system executing computer-readable instructions, code, and/or programs to function differently depending upon the values of the variables or other data structures that are provided. As referred to herein a “processing system” may comprise a computing device, or computing system, including one or more processors, or cores (e.g., as illustrated in FIG. 4 and discussed below) or multiple computing devices collectively configured to perform various steps, functions, and/or operations in accordance with the present disclosure.

In one example, telecommunication network 102 may also include an application server (AS) 104 and one or more databases (DBs) 106. In one example, the application server 104 may comprise a computing device or processing system, such as computing system 400 depicted in FIG. 4, and may be configured to perform one or more steps, functions, or operations for reserving a network-connected storage locker having at least one sterilization component that is capable of fulfilling a sterilization requirement and that is available for assignment for an exchange of an item (e.g., as illustrated in FIG. 3 and described below). In one example, DB(s) 106 may comprise physical storage device(s) integrated with AS 104 (e.g., a database server), or attached or coupled to AS 104, to store various types of information in support of systems for reserving a network-connected storage locker having at least one sterilization component that is capable of fulfilling a sterilization requirement and that is available for assignment for an exchange of an item. For instance, DB(s) 106 may obtain and store the same or similar information as DB(s) 118.

An example network-connected storage locker 190 may include a number of components, such as one or more processors 197, input/output (I/O) devices, such as a display screen, a touchpad and/or keypad, an optical scanner and/or outward facing camera, an interior camera, etc., and a door 196, which may be controlled by the processor(s) 197 in conjunction with inputs from the I/O devices. In accordance with the present disclosure, network-connected storage locker 190 may further include one or more sterilization components 199, which may comprise, for example: an ultraviolet light source component, an ozone generator component, a steam sterilization component (which in one example may be for flash steam sterilization), an ethylene oxide sterilization component, a gas plasma sterilization component (e.g., for generating a hydrogen peroxide gas plasma), a microwave radiation source, an infrared radiation source, and so forth. The network-connected storage locker may include other components (not shown), such as computing memory and storage, one or more batteries, a power cable and/or solar power unit, an antenna and wireless transceiver (and/or wired networking components), a refrigeration unit, a heating unit, power connectors, and so forth. In one example, the network-connected storage locker may comprise a computing device or processing system, such as computing system 400 depicted in FIG. 4, and may be configured to perform one or more steps, functions, or operations for reserving a network-connected storage locker having at least one sterilization component that is capable of fulfilling a sterilization requirement and that is available for assignment for an exchange of an item (e.g., as illustrated in FIG. 3 and described below). In addition, each of network-connected storage lockers 191-194 may have a same or similar components and may provide the same or similar functions as network-connected storage locker 190. However, for ease of illustration, various details of network-connected storage lockers 191-194 are omitted from FIG. 1.

In an illustrative example, server(s) 116 may provide a network-connected storage locker management and/or reservation system. For instance, network-connected storage lockers may be provided and managed by such a system, or may be offered for service by various owners, lessees, or other entities which may register the network-connected storage lockers with server(s) 116. A user 140 may have an item 180 that the user 140 has agreed to exchange with another party, e.g., user 142, such as by way of sale, donation, barter, etc. The agreement for the exchange may take place via any number of different channels, such as via an online marketplace, via a verbal agreement of the parties, etc. The user 140, via device 110 may notify server(s) 116 of the exchange, and may request a network-connected storage locker for such an exchange. The user 140 may provide an item description, the dimensions of the item 180, the weight of the item 180, the recipient party (e.g., user 142) and/or an identifier of the device of the recipient party (e.g., device 112), and/or any time requirements for the exchange (e.g., must be available within 24 hours, 48 hours, etc., must be available to hold the item for no less than 48 hours, no less than 72 hours, etc., and so forth). The user 140 may also provide additional requirements for the network-connected storage locker for the exchange, such as being no more than a certain distance from user 140, having less than a maximum cost the user 140 is willing to pay, and having various additional features or capabilities. For instance, in accordance with the present disclosure, user 140 may specify a sterilization (or “sanitation”) requirement. Similarly, the user 140 may specify that an interior camera (e.g., one of the I/O devices 198) be available for verification of the item 180 being stored in the network-connected storage locker is also required and/or desired. With respect to the sterilization requirement, the user 140 may specify one or more types of sterilization that are acceptable. Alternatively, or in addition, the user 140 may request sterilization, where the server(s) 116 may select or may recommend a type of sterilization based upon the type of item to be stored from the item description.

In one example, the user 142 may alternatively or additionally request and/or specify requirements for the exchange of item 180 via a network-connected storage locker. For instance, the user 140 may not have a sterilization requirement. However, the user 142 who is the recipient of the item 180 may have a greater desire that the item 180 be sterilized. Thus, if user 140 has not already included a sterilization requirement, then user 142 may make such a request to server(s) 116. User 142 may similarly specify other requirements or requests, such as for cold storage, for a longer duration of storage, for an interior camera that is network-accessible to verify the contents of the network-connected storage locker, and so forth.

The server(s) 116 may receive any and all of such information pertaining to the exchange of item 180 and may then locate and select an available network-connected storage locker for assigning to the exchange of item 180. For instance, server(s) 116 may track locations of user 140 and user 142 (having received the users' express consent to do so), such as via devices 110 and 112, and may then query a network-connected storage locker database from DB(s) 118 for available network-connected storage lockers that are: most equidistant from users 140 and 142, have the requisite capacity by volume and/or weight, have the requisite additional capabilities, including at least a sanitization capability, and so forth. In one example, server(s) 116 may select from among different available network-connected storage lockers meeting the requirements(s) using any number of algorithmic criteria, such as weighting a distance factor, a cost factor, an availability factor (e.g., favoring network-connected storage units that are available for a longer duration of time before the item is expected to vacate), and so forth. In an example where multiple network-connected storage lockers with the same or similar capabilities are available at a same location, server(s) 116 may select a network-connected storage locker in a round-robin fashion, in accordance with a least recently used (LRU) rule, and so forth. It should be noted that in one example, user 140 or user 142 may specify a required type of sanitation, or one or more acceptable types of sanitization. However, in another example, the user 140 or user 142 may request “sanitization” as a requirement, wherein the server(s) 116 may select acceptable types of sanitization based upon the item description (e.g., UV light may not be used for an item of vintage clothing, whereas ozone may be avoided if the description is computer or electronics, and so on). In one example, the server(s) 116 may give a warning that the system is not responsible for damaged items if the user chooses to allow the server(s) 116 to select a type of sanitization. For instance, an incomplete or inaccurate description may lead server(s) 116 to select a method of sanitization that may not be appropriate for the item type. In one example, the server(s) 116 may utilize a matching table that associates different item types with acceptable (and/or unacceptable) types of sanitization.

In this present example, server(s) 116 may select network-connected storage locker 190 and may notify user 140 and user 142 of the selection, e.g., by communications to devices 110 and 112. In one example, server(s) 116 may provide respective access tokens (or “security tokens”) to devices 110 and 112, such as QR codes to be scanned at network-connected storage locker 190, a password/passcode, etc. In addition, server(s) 116 may provide information associated with the access tokens to network-connected storage locker 190, such as public keys to match private keys comprising the access tokens given to devices 110 and 112, or the passwords/passcodes, etc. In another example, network-connected storage locker 190 may generate access tokens for users 140 and 142, which may be transmitted to server(s) 116 when network-connected storage locker 190 is assigned to the exchange of item 180. In one example, server(s) 116 may select network-connected storage locker 190, and may transmit a request to network-connected storage locker 190 to accept the assignment. Network-connected storage locker 190 may then respond to accept/confirm (or to deny the request). For instance, as noted above, network-connected storage lockers may be provided by various entities for public use and may be registered with server(s) 116 for assignment to be used for exchange of items. For example, one entity may elect to disallow devices using lithium-ion batteries to be stored in its network-connected storage lockers due to potential fire hazard and so on.

In one example, server(s) 116 may update a network-connected storage locker database of DB(s) 118 to record the assignment/reservation of network-connected storage locker 190. In one example, the record may include a time window for the assignment. For instance, the assignment may not necessarily begin at the time the reservation is made, but may be for a time period starting in the future, such as starting 48 hours in the future, 72 hours in the future, etc. In addition, the time window may be to reserve the network-connected storage unit for 24 hours, 48 hours, 72 hours, etc. after the time window begins. In one example, server(s) 116 may also update a transaction/item exchange database of DB(s) 118 to include a recordation of the assignment of network-connected storage locker to the transaction/exchange associated with item 180. In another example, server(s) 116 may transmit, to another system, a notification of the assignment of network-connected storage locker 190 to the exchange/transaction. For instance, the other system may comprise a separate online marketplace or other systems via which the exchange has been facilitated.

At some time, user 140 may travel to the location of network-connected storage locker 190, and may utilize the access token to cause network-connected storage locker 190 to open the door 196. For instance, a QR code may be presented via a display screen of device 110 that may be scanned by an optical scanner/camera of I/O device 198. Network-connected storage locker 190 may then open door 196 when the QR code matches an expected QR code for the reservation of the network-connected storage locker 190, e.g., during the designated time window of the reservation. In another example, user 140 may enter a password/passcode via a keypad or touchscreen of I/O device 198. In any case, network-connected storage locker 190 (e.g., processor(s) 197 thereof) may cause door 196 to open when the correct access token is presented. In one example, network-connected storage locker 190 may transmit a notification to server(s) 116 that network-connected storage locker 190 has been opened with the access token given to user 140. In one example, user 140 may place item 180 inside the network-connected storage locker 190 and close the door 196, which may then automatically lock. In one example, an interior camera of network-connected storage locker 190 may capture one or more images (e.g., one or more still images, or a video clip) of the item 180 inside. The network-connected storage locker 190 may then transmit a notification to server(s) 116 that network-connected storage locker 190 has been locked with the item inside, where the notification may include the one or more images. Similar to the above, the server(s) 116 may update the status of the network-connected storage locker 190 to “in use.”

As noted above, a requirement for the exchange/transaction may be for sterilization of the item 180 by the network-connected storage locker 190. Accordingly, the network-connected storage locker 190 may activate and utilize one or more of the sterilization component(s) 199 when the item 180 is locked inside the network-connected storage locker 190. In one example, the one or more of sterilization component(s) 199 may be activated as soon as practicable after the item 180 is placed in the network-connected storage locker 190. For instance, it may be unknown when user 142 will come to retrieve the item 180, and thus, it may be beneficial to immediately sterilize the item 180 in case that the user 142 arrives very soon after user 140 having placed item 180 in the network-connected storage locker 190. In another example, the exchange/transaction may have a built-in minimum time delay between when the item 180 is placed in the network-connected storage locker 190 and when the item 180 is removed. For instance, the item 180 may be removed no earlier than 12 hours after being placed in the network-connected storage locker. In this case, the network-connected storage locker 190 may select a time within this window to conduct the sterilization, or may conduct multiple rounds of applying the sterilization component(s) 199 during this time window. For instance, this may further increase the likelihood that any pathogens are sterilized or eradicated.

Continuing with the present example, at some later time, user 142 may travel to the location of network-connected storage locker 190, and may utilize the access token given to user 142 to cause network-connected storage locker 190 to open the door 196. In one example, network-connected storage locker 190 may transmit a notification to server(s) 116 that network-connected storage locker 190 has been opened with the access token given to user 142. In one example, user 142 may extract the item 180 and close the door 196, which may then automatically lock. In one example, an interior camera of network-connected storage locker 190 may capture one or more images (e.g., one or more still images, or a video clip) of the empty network-connected storage locker 190 and/or of the act of user 142 removing the item 180. The network-connected storage locker 190 may then transmit a notification to server(s) 116 that network-connected storage locker 190 has been emptied and locked, where the notification may include the one or more images. Similar to the above, the server(s) 116 may update the status of the network-connected storage locker 190 to “vacant” in the network-connected storage locker database of DB(s) 118.

In one example, server(s) 116 may also update a transaction/item exchange database of DB(s) 118 to include a recordation of the completion of the transaction/exchange associated with item 180. In another example, server(s) 116 may transmit, to another system, a notification of the completion of the use of network-connected storage locker 190 in connection with the exchange/transaction. For instance, the other system may comprise a separate online marketplace or other systems via which the exchange has been facilitated.

It should be noted that the foregoing is just one illustrative example of how examples of the present disclosure for reserving a network-connected storage locker having at least one sterilization component that is capable of fulfilling a sterilization requirement and that is available for assignment for an exchange of an item may operate. Thus, it should be noted that in other, further, and different examples, aspects described above with respect to server(s) 116 may alternatively or additionally be provided by AS 104. For instance, an operator of telecommunication network 102 may provide a network-based and/or cloud-based network-connected storage locker management and/or reservation system, e.g., via AS 104, or AS 104 and DB 106. In another example, user 140 may wish to donate or may not have a buyer for item 180, but may wish to put item 180 into a publicly accessible space for being obtained by others. In addition, the user 140 may wish to entice a buyer or other acquirers by offering the sanitization of item 180 as part of the exchange. As such, the sanitization requirement may be included in a request from user 140 to server(s) 116 to reserve a network-connected storage locker for the item 180. As noted above, the present disclosure may identify a best location for placing the item 180 which may help to result in a sale (or a fast acquisition, if the manner of exchange is not necessarily a sale and purchase). For instance, server(s) 116 may access past purchase histories, wishlist data, watch lists, or the like to identify users who are interested in the same type or similar type of item as item 180, may identify a location or area with the most users having the interest are located (e.g., by clustering such users according to a clustering algorithm and determining a central location of a largest cluster), may identify network-connected storage lockers at or near the location, may select one of the network-connected storage lockers, and so forth. The user 140 may then be directed to a selected network-connected storage locker, such as network-connected storage locker 191, and may be provided with an access token. The user 140 may access and place item 180 in network-connected storage locker 191, which may notify server(s) 116 that the item 180 is present (wherein in one example, the notification may include one or more images of the item 180 in the network-connected storage locker 191). Server(s) 116 may then notify all or a portion of the interested users in the area that item 180 is available, may provide image(s) or a video clip of the item 180 in the network-connected storage locker 191, may provide information on the sanitization status of the item 180 (e.g., recently UV sanitized) or the availability of a sanitization service via one or more sanitization components of network-connected storage locker 191, and so on.

In another example, a location may have multiple network-connected storage lockers that may be controlled by a centralized computing system that may manage a plurality of network-connected storage lockers. For instance, such a centralized computing system may receive inputs from I/O devices of respective network-connected storage lockers, may determine if access tokens are as expected, may allow doors of network-connected storage lockers to open when valid access tokens are presented, may communicate with server(s) 116, and so forth.

It should also be noted that the system 100 has been simplified. Thus, the system 100 may be implemented in a different form than that which is illustrated in FIG. 1, or may be expanded by including additional endpoint devices, access networks, network elements, application servers, etc. without altering the scope of the present disclosure. In addition, system 100 may be altered to omit various elements, substitute elements for devices that perform the same or similar functions, combine elements that are illustrated as separate devices, and/or implement network elements as functions that are spread across several devices that operate collectively as the respective network elements. For example, the system 100 may include other network elements (not shown) such as border elements, routers, switches, policy servers, security devices, gateways, a content distribution network (CDN) and the like. For example, portions of telecommunication network 102 and/or access networks 120 and 122 may comprise a content distribution network (CDN) having ingest servers, edge servers, and the like. Similarly, although only two access networks 120 and 122 are shown, in other examples, access networks 120 and/or 122 may each comprise a plurality of different access networks that may interface with telecommunication network 102 independently or in a chained manner. For example, device 113 and server 116 may access telecommunication network 102 via different access networks, devices 110 and 112 may access telecommunication network 102 via different access networks, and so forth. Thus, these and other modifications are all contemplated within the scope of the present disclosure.

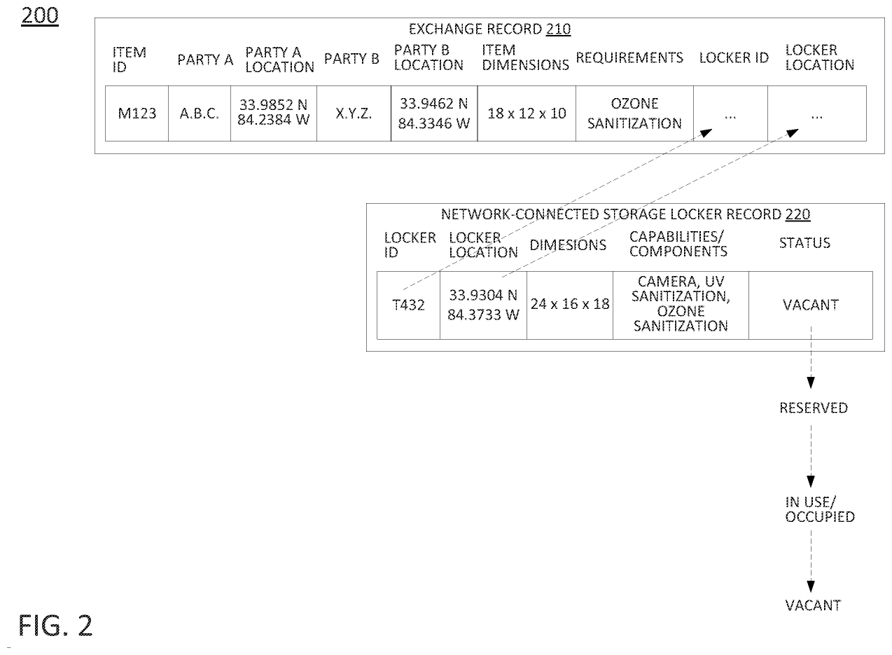

FIG. 2 illustrates an example of an exchange record 210 and an example network-connected storage locker record 220 relating to examples of the present disclosure. The examples of FIG. 2 may be related to various components as illustrated in FIG. 1 and discussed above. For instance, the exchange record 210 may be stored by an exchange/transaction database of DB(s) 118 or DB 106. Similarly, network-connected storage locker record 220 may be stored by a network-connected storage locker database of DB(s) 118 or DB 106. As illustrated in FIG. 2, exchange record 210 includes an item identifier (ID), which may be a predefined identifier, or which may be assigned when a user (party A) begins an exchange/transaction associated with the item. The exchange record 210 may further include the identification of party A (e.g., the name of party A) and the location of party A (e.g., as determined from a mobile device of party A). The exchange record 210 may further include the identification of party B (e.g., the name of party B) and the location of party B (e.g., as determined from a mobile device of party B).

As further illustrated in FIG. 2, the exchange record 210 may also include item dimensions, which may be input by party A, or which may be calculated from one or more images of the item provided by party A. In the example of FIG. 2, the exchange record 210 may include fields for a locker identifier (ID) and a locker location of a network-connected storage locker that will be assigned to the exchange. When the network-connected storage locker is selected and/or confirmed, the details may then be added to these respective fields.

In this regard, network-connected storage locker record 220 may be one of a number of network-connected storage locker records in a network-connected storage locker database. The network-connected storage locker record 220 is for a network-connected storage locker having a locker identifier (ID) of T432, which may be assigned to the exchange/transaction associated with exchange record 210. The network-connected storage locker record 220 may include dimensions of the network-connected storage locker, the components/capabilities of the network-connected storage locker, and a status of the network-connected storage locker. The locker location and any or all of these fields may be used to identify the network-connected storage locker as being available and meeting the requirements/criteria of the exchange/transaction associated with exchange record 210. For instance, exchange record 210 indicates the item dimensions and a requirement for ozone sanitization. Network-connected storage locker record 220 indicates the dimensions of the network-connected storage locker (which exceeds the dimensions of the item, and which therefore has sufficient capacity to store the item therein) and also indicates that the network-connected storage locker includes ozone sanitization components (which meets the requirements of the transaction/exchange indicated in the exchange record 210). In addition, the network-connected storage locker record 220 indicates that the network-connected storage locker is vacant, and thus available for assignment to the exchange/transaction. As such, the network-connected storage locker having locker ID T432 may be assigned to the exchange/transaction associated with exchange record 210.

When the network-connected storage locker having locker ID T432 is assigned to the exchange/transaction, or when the assignment is confirmed by the network-connected storage locker, the status may be changed to “reserved” in the network-connected storage locker record 220. At a later time, when party A accesses the network-connected storage locker and places the item inside, the status may be changed to “in use” or “occupied” in the network-connected storage locker record 220. Similarly, at a later time when party B accesses the network-connected storage locker to retrieve the item inside, the status may be changed back to “vacant” in the network-connected storage locker record 220. It should be noted that FIG. 2 illustrates just one example of each of an exchange record 210 and a network-connected storage locker record 220, and that in other, further, and different examples exchange records and network-connected storage locker records of the present disclosure may have a different form and may include more or less fields. For instance, exchange record 210 may include an item description field, an exchange/transaction status field, etc. The network-connected storage locker record 220 may include a reservation time window field, a cost field, a passcode field, and so on. Thus, these and other modifications are all contemplated within the scope of the present disclosure.

FIG. 3 illustrates a flowchart of an example method 300 for reserving a network-connected storage locker having at least one sterilization component that is capable of fulfilling a sterilization requirement and that is available for assignment for an exchange of an item. In one example, the method 300 is performed by a component of the system 100 of FIG. 1, such as by server(s) 116, application server 104, and/or any one or more components thereof (e.g., a processor, or processors, performing operations stored in and loaded from a memory), or by server(s) 116 or application server 104, in conjunction with one or more other devices, such as DB(s) 106, DB(s) 118, devices 110 and 112, network-connected storage lockers 190-194, and so forth. In one example, the steps, functions, or operations of method 300 may be performed by a computing device or system 400, and/or processor 402 as described in connection with FIG. 4 below. For instance, the computing device or system 400 may represent any one or more components of application server 104, and so forth in FIG. 1 that is/are configured to perform the steps, functions and/or operations of the method 300. Similarly, in one example, the steps, functions, or operations of method 300 may be performed by a processing system comprising one or more computing devices collectively configured to perform various steps, functions, and/or operations of the method 300. For instance, multiple instances of the computing device or processing system 400 may collectively function as a processing system. For illustrative purposes, the method 300 is described in greater detail below in connection with an example performed by a processing system. The method 300 begins in step 305 and proceeds to step 310.

At step 310, the processing system obtains information regarding an exchange of an item, the exchange having a sterilization requirement. In one example, step 310 may comprise obtaining a notification from a computing system associated with a source of the item. In one example, step 310 may include obtaining a notification of the sterilization requirement for the exchange. For instance, the notification of the sterilization requirement may be included in the notification of the exchange, or may be a separate communication. The notification of the sterilization requirement may be obtained, for example, from the computing system associated with the source of the item or a computing system associated with another party to the exchange of the item (e.g., a buyer, or other person or entity that will retrieve the item). In this regard, it should be noted that in one example, step 310 may include obtaining an identification of the source of the item and/or an identification of a party to retrieve the item. For instance, in one example, both parties to the exchange may be identified in a notification obtained from a computing system associated with the source of the item. However, in another example, the party to retrieve the item may be identified at a later time. In one example, the processing system may include an online marketplace that may facilitate or broker the exchange, and which may learn the identities of both parties. Alternatively, the exchange may be agreed upon via another online marketplace, where the party to retrieve the item may be made know to the processing system at a later time. In one example, the identity of the party to retrieve the item and the sterilization requirement may be obtained as part of a same communication. In one example, step 310 may also include obtaining a location of the source of the item, a location of the party to retrieve the item, or both.

At optional step 315, the processing system may determine at least one type of sterilization component that is capable of fulfilling the sterilization requirement. For instance, the processing system may make a determination based upon a composition of the item (e.g., UV light acceptable for electronics, ozone unacceptable for electronics, ozone and UV acceptable for sports equipment, etc.). In one example, the at least one type of sterilization component is determined from among various types of sterilization components that are available from a plurality of storage lockers. For instance, the types of sterilization components may include an ultraviolet light source component, an ozone generator component, a steam sterilization component (which in one example may be for flash steam sterilization), an ethylene oxide sterilization component, a gas plasma sterilization component (e.g., for generating a hydrogen peroxide gas plasma), a microwave radiation source, an infrared radiation source, and so forth.

At step 320, the processing system identifies a network-connected storage locker having at least one sterilization component that is capable of fulfilling the sterilization requirement and that is available for assignment to the exchange (e.g., for storing the item in connection with the exchange). In one example, the at least one sterilization component is of a type that may be determined at optional step 315. In one example, step 320 may include determining a potential geographical area where a network-connected storage locker can be selected for the exchange based upon at least one: of a location of the source of the item, or a location of a party to retrieve the item. In one example, step 320 may include determining an area of the network-connected storage locker based upon a location of at least one prospective recipient of the item. For instance, the at least one prospective recipient may have indicated an interest in the item or a type of item of the item (e.g., in a wishlist, user profile, past purchases or other transactions/exchanges, etc.). The at least one prospective recipient could include (but need not include) the party to retrieve the item. For example, a buyer not in the selected area could be more interested such that the buyer is more immediately willing to travel further to the network-connected storage locker to obtain the item than any of the at least one prospective buyer.

At step 325, the processing system transmits a first notification to the network-connected storage locker of a reservation of the network-connected storage locker for the exchange.

At optional step 330, the processing system may transmit, to the network-connected storage locker, information associated with a security token (or “access token”) for accessing the network-connected storage locker. For instance, the information associated with the security token may comprise the security token itself or a copy thereof, e.g., a passcode/password, a QR code, or the like, or can be a public key associated with a private key, or the like. In one example, the information associated with the security token may be transmitted via the first notification (e.g., as part of the first notification). In one example, optional step 330 may include transmitting to the network-connected storage locker, information associated with a second security token for accessing the network-connected storage locker. For instance, the first security token may be for the owner of the item to access the network-connected storage locker, while the second security token may by for the party to retrieve the item to gain access to the network-connected storage locker. In one example, security token(s) may instead by provided by the network-connected storage locker, in which case optional step 330 may be omitted.

At optional step 335, the processing system may obtain a confirmation from the network-connected storage locker of an acceptance of the reservation. For instance, in an example where the network-connected storage locker is controlled by another entity, the processing system may not have a guarantee that the network-connected storage locker will be available for the item until such a confirmation is provided by the network-connected storage locker and obtained by the processing system.

At optional step 340, the processing system may obtain a (first) security token for accessing the network-connected storage locker. In one example, optional step 340 may include obtaining a second security token for accessing the network-connected storage locker. For instance, the first security token may be for the owner of the item to access the network-connected storage locker, while the second security token may by for the party to retrieve the item to gain access to the network-connected storage locker. In one example, the security token(s) may be obtained as part of the confirmation from the network-connected storage locker. Optional step 340 may be performed in an example in which the processing system does not itself select or generate the security token(s).

At step 345, the processing system transmits to the computing system associated with the source or owner of the item a second notification of the reservation of the network-connected storage locker for the exchange. In one example, the second notification of the reservation of the network-connected storage locker for the exchange includes the security token.

At optional step 350, the processing system may transmit, to a computing system associated with the party to retrieve the item, a third notification of the reservation of the network-connected storage locker for the exchange. In one example, optional step 350 may include providing the second security token to the computing system associated with the party to retrieve the item, for accessing the network-connected storage locker.

At optional step 355, the processing system may transmit, to the network-connected storage locker, information associated with the second security token for accessing the network-connected storage locker. For instance, optional step 355 may be performed when the processing system selects or generates the second security token. The information associated with the second security token may comprise the security token itself or a copy thereof, e.g., a passcode/password, a QR code, or the like, or can be a public key associated with a private key, or the like. In one example, the information associated with the security token may be transmitted via the first notification (e.g., as part of the first notification). It should be noted that in one example, the second security token may be provided at optional step 330. However, in another example, the second security token may be provided at a later time (e.g., at optional step 355). For instance, the source or owner of the item may begin an exchange/transaction where the party to retrieve the item is unknown. The item may be placed in the network-connected storage locker, and then at some later time, a buyer or another party to retrieve the item may be identified. Thus, at such time, a second security token may be selected or generated, and provided to the party to retrieve the item. In addition, in an example in which the processing system selects or generates the second security token, the information associated with the second security token may also be provided to the network-connected storage locker (e.g., at optional step 355).

At optional step 360, the processing system may obtain at least one image of the item in the network-connected storage locker. For instance, the network-connected storage locker may include a camera to obtain one or more images (which may include still images and/or video) of the item inside the network-connected storage locker, where the network-connected storage locker may transmit the one or more images to the processing system.

At optional step 365, the processing system may transmit the at least one image to at least one of the computing system associated with the source or owner of the item, or a computing system associated with the party to retrieve the item. Alternatively, or in addition, the at least one image may be transmitted to one or more prospective recipients (e.g., buyers, members of a sharing or bartering community, etc.), e.g., where the item is not yet sold or where the party to retrieve the item is otherwise not yet identified. In addition, the one or more prospective recipients may have indicated an interest to receive additional information relating to the item, such as price drops, sterilization status changes, additional images, etc.

At optional step 370, the processing system may obtain a notification of a completion of a sterilization process for the item via the network-connected storage locker.

At optional step 375, the processing system may transmit a confirmation of the completion of the sterilization process to at least one of the computing system associated with the source of the item or a computing system associated with a party to retrieve the item. Alternatively, or in addition, the confirmation of the completion of the sterilization may be transmitted to one or more prospective recipients (e.g., buyers, members of sharing or bartering community, etc.), e.g., where the item is not yet sold or where the party to retrieve the item is otherwise not yet identified. For instance, one or more prospective recipients may have received a notification of the item being available and located in the network-connected storage locker. In addition, the one or more prospective recipients may have indicated an interest to receive additional information relating to the item, such as price drops, sterilization status changes, additional images, etc.

Following step 345 or any of optional steps 350-375, the method 300 proceeds to step 395 where the method 300 ends.

It should be noted that the method 300 may be expanded to include additional steps, or may be modified to replace steps with different steps, to combine steps, to omit steps, to perform steps in a different order, and so forth. For instance, in one example the processing system may repeat one or more steps of the method 300, such as obtaining and transmitting notifications of additional rounds of sanitization, obtaining new image(s) from a camera of the network-connected storage locker, and so forth. For example, the item may sit in the network-connected storage locker for some time without a buyer or another party to retrieve the item. Thus, the item may be resanitized, e.g., periodically or otherwise. In another example, the method 300 may include an additional step(s) of selecting or generating the first security token and/or the second security token. In another example, the method 300 may include facilitating an agreement between the source of the item and the party to retrieve the item, such as presenting a listing for the item via a website, obtaining an offer to purchase the item, obtaining an acceptance of the offer, coordinating payment and/or confirming payment, confirming that the item is retrieved from the network-connected storage locker, e.g., by a purchaser or other party, releasing a payment held in escrow in response to the retrieval, and so forth. In one example, step 310 may include obtaining addition requirements for the exchange of the item, such as a refrigeration and/or cooling requirement, and so forth. In such case, optional step 320 may include identifying the network-connected storage locker having the at least one sterilization component that is capable of fulfilling the sterilization requirement, wherein the network-connected storage locker is also identified as being capable of fulfilling any other requirements, such as the refrigeration and/or cooling requirement.

In one example, the method 300 may be expanded or modified to include additional, other, further, and/or different steps, functions, and/or operations as described elsewhere herein. In yet another example, the method 300 may comprise a network-connected storage locker (e.g., a processing system thereof) performing operations complementary to those set forth above (e.g., providing status information to a network-based system, obtaining notifications and/or requests for assignment for holding an item to be exchanged, receiving and/or providing security tokens, granting access and relocking the network-connected storage container after accesses by the source of the item and a party to retrieve the item, obtaining and transmitting one or more images of the item in the network-connected storage container, sanitizing the item via one or more sanitization components, detecting access of the network-connected storage container by the retrieving party, sending a confirmation that the item has been retrieved (e.g., sending an image of the party retrieving the item), self-sterilizing between uses/placement of items therein, and so on). Thus, these and other modifications are all contemplated within the scope of the present disclosure.

In addition, although not expressly specified above, one or more steps of the method 300 may include a storing, displaying and/or outputting step as required for a particular application. In other words, any data, records, fields, and/or intermediate results discussed in the method(s) can be stored, displayed and/or outputted to another device as required for a particular application. Furthermore, operations, steps, or blocks in FIG. 3 that recite a determining operation or involve a decision do not necessarily require that both branches of the determining operation be practiced. In other words, one of the branches of the determining operation can be deemed as an optional step. Furthermore, operations, steps or blocks of the above described method(s) can be combined, separated, and/or performed in a different order from that described above, without departing from the example embodiments of the present disclosure.

FIG. 4 depicts a high-level block diagram of a computing device or processing system specifically programmed to perform the functions described herein. For example, any one or more components or devices illustrated in FIG. 1 or described in connection with the examples of FIG. 2 or 3 may be implemented as the processing system 400. As depicted in FIG. 4, the processing system 400 comprises one or more hardware processor elements 402 (e.g., a microprocessor, a central processing unit (CPU) and the like), a memory 404, (e.g., random access memory (RAM), read only memory (ROM), a disk drive, an optical drive, a magnetic drive, and/or a Universal Serial Bus (USB) drive), a module 405 for reserving a network-connected storage locker having at least one sterilization component that is capable of fulfilling a sterilization requirement and that is available for assignment for an exchange of an item, and various input/output devices 406, e.g., a camera, a video camera, storage devices, including but not limited to, a tape drive, a floppy drive, a hard disk drive or a compact disk drive, a receiver, a transmitter, a speaker, a display, a speech synthesizer, an output port, and a user input device (such as a keyboard, a keypad, a mouse, and the like).

Although only one processor element is shown, it should be noted that the computing device may employ a plurality of processor elements. Furthermore, although only one computing device is shown in the Figure, if the method(s) as discussed above is implemented in a distributed or parallel manner for a particular illustrative example, i.e., the steps of the above method(s) or the entire method(s) are implemented across multiple or parallel computing devices, e.g., a processing system, then the computing device of this Figure is intended to represent each of those multiple computing devices. Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented. The hardware processor 402 can also be configured or programmed to cause other devices to perform one or more operations as discussed above. In other words, the hardware processor 402 may serve the function of a central controller directing other devices to perform the one or more operations as discussed above.

It should be noted that the present disclosure can be implemented in software and/or in a combination of software and hardware, e.g., using application specific integrated circuits (ASIC), a programmable logic array (PLA), including a field-programmable gate array (FPGA), or a state machine deployed on a hardware device, a computing device, or any other hardware equivalents, e.g., computer readable instructions pertaining to the method(s) discussed above can be used to configure a hardware processor to perform the steps, functions and/or operations of the above disclosed method(s). In one example, instructions and data for the present module or process 405 for reserving a network-connected storage locker having at least one sterilization component that is capable of fulfilling a sterilization requirement and that is available for assignment for an exchange of an item (e.g., a software program comprising computer-executable instructions) can be loaded into memory 404 and executed by hardware processor element 402 to implement the steps, functions or operations as discussed above in connection with the example method 300. Furthermore, when a hardware processor executes instructions to perform “operations,” this could include the hardware processor performing the operations directly and/or facilitating, directing, or cooperating with another hardware device or component (e.g., a co-processor and the like) to perform the operations.

The processor executing the computer readable or software instructions relating to the above described method(s) can be perceived as a programmed processor or a specialized processor. As such, the present module 405 for reserving a network-connected storage locker having at least one sterilization component that is capable of fulfilling a sterilization requirement and that is available for assignment for an exchange of an item (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. Furthermore, a “tangible” computer-readable storage device or medium comprises a physical device, a hardware device, or a device that is discernible by the touch. More specifically, the computer-readable storage device may comprise any physical devices that provide the ability to store information such as data and/or instructions to be accessed by a processor or a computing device such as a computer or an application server.

While various embodiments have been described above, it should be understood that they have been presented by way of example only, and not limitation. Thus, the breadth and scope of a preferred embodiment should not be limited by any of the above-described example embodiments, but should be defined only in accordance with the following claims and their equivalents.

What is claimed is:

1. A method comprising:
obtaining, by a processing system including at least one processor, information regarding an exchange of an item, the exchange having a sterilization requirement for the exchange;
identifying, by the processing system, a network-connected storage locker having at least one sterilization component that is capable of fulfilling the sterilization requirement and that is available for assignment to the exchange;
transmitting, by the processing system, a first notification to the network-connected storage locker of a reservation of the network-connected storage locker for the exchange; and
transmitting, by the processing system, to a computing system associated with a source of the item a second notification of the reservation of the network-connected storage locker for the exchange.

2. The method of claim 1, further comprising:
obtaining a confirmation from the network-connected storage locker of an acceptance of the reservation.

3. The method of claim 1, further comprising:
obtaining a security token for accessing the network-connected storage locker, wherein the second notification of the reservation of the network-connected storage locker for the exchange includes the security token.

4. The method of claim 1, further comprising:
transmitting, to the network-connected storage locker, information associated with a security token for accessing the network-connected storage locker, wherein the second notification of the reservation of the network-connected storage locker for the exchange includes the security token.

5. The method of claim 4, wherein the information associated with the security token is transmitted via the first notification.

6. The method of claim 1, further comprising:
determining at least one type of sterilization component that is capable of fulfilling the sterilization requirement, wherein the at least one sterilization component is of the at least one type of sterilization component.

7. The method of claim 6, wherein the determining the at least one type of sterilization component is based upon a composition of the item.

8. The method of claim 6, wherein the at least one type of sterilization component is determined from among a plurality of types of sterilization components that are available from a plurality of network-connected storage lockers, the plurality of network-connected storage lockers including the network-connected storage locker.

9. The method of claim 8, wherein the plurality of types of sterilization components comprises at least two of:
an ultraviolet light source component;
an ozone generator component;
a steam sterilization component;
an ethylene oxide sterilization component;
a gas plasma sterilization component;
a microwave radiation source; or
an infrared radiation source.

10. The method of claim 1, wherein the obtaining the information regarding the exchange of the item comprises:
obtaining a notification of the sterilization requirement for the exchange.

11. The method of claim 10, wherein the notification of the sterilization requirement is obtained from the computing system associated with the source of the item or a computing system associated with another party to the exchange of the item.

12. The method of claim 1, wherein the obtaining the information regarding the exchange of the item comprises:
obtaining an identification of a party to retrieve the item.

13. The method of claim 12, further comprising:
transmitting to a computing system associated with the party to retrieve the item a third notification of the reservation of the network-connected storage locker for the exchange.

14. The method of claim 13, wherein the transmitting the third notification comprises:
providing a second security token to the computing system associated with the party to retrieve the item, for accessing the network-connected storage locker.

15. The method of claim 14, further comprising:
transmitting, to the network-connected storage locker, information associated with the second security token for accessing the network-connected storage locker.

16. The method of claim 12, wherein the identifying the network-connected storage locker comprises determining an area of the network-connected storage locker based upon at least one of:
a location of the source of the item; or
a location of the party to retrieve the item; or
a location of at least one prospective recipient of the item.

17. The method of claim 12, further comprising:
obtaining at least one image of the item in the network-connected storage locker; and
transmitting the at least one image to at least one of: the computing system associated with the source of the item, or a computing system associated with the party to retrieve the item.

18. The method of claim 1, further comprising:
obtaining a notification of a completion of a sterilization process for the item via the network-connected storage locker; and
transmitting a confirmation of the completion of the sterilization process to at least one of: the computing system associated with the source of the item or a computing system associated with a party to retrieve the item.

19. A non-transitory computer-readable medium storing instructions which, when executed by a processing system including at least one processor, cause the processing system to perform operations, the operations comprising:
obtaining information regarding an exchange of an item, the exchange having a sterilization requirement for the exchange;
identifying a network-connected storage locker having at least one sterilization component that is capable of fulfilling the sterilization requirement and that is available for assignment to the exchange;
transmitting a first notification to the network-connected storage locker of a reservation of the network-connected storage locker for the exchange; and
transmitting to a computing system associated with a source of the item a second notification of the reservation of the network-connected storage locker for the exchange.

20. An apparatus comprising:
a processing system including at least one processor; and
a computer-readable medium storing instructions which, when executed by the processing system, cause the processing system to perform operations, the operations comprising:
obtaining information regarding an exchange of an item, the exchange having a sterilization requirement for the exchange;
identifying a network-connected storage locker having at least one sterilization component that is capable of fulfilling the sterilization requirement and that is available for assignment to the exchange;
transmitting a first notification to the network-connected storage locker of a reservation of the network-connected storage locker for the exchange; and
transmitting to a computing system associated with a source of the item a second notification of the reservation of the network-connected storage locker for the exchange.
